(19) United States
(12) Patent Application Publication
(10) Pub. No.: US 2008/0200967 A1
(43) Pub. Date: Aug. 21, 2008

(54) Apparatus and Method For Electrostimulation /Sensing in Vivo

(75) Inventors: Youri Ponomarev, Leuven (BE); Matthias Merz, Leuven (BE); Remco Pijnenburg, Hoogeloon (NL)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 12/066,690

(22) PCT Filed: Sep. 13, 2006
(86) PCT No.: PCT/IB06/53259

§ 371 Date: Mar. 13, 2008

Related U.S. Application Data
(60) Provisional application No. 60/717,475, filed on Sep. 15, 2005.

(51) Int. Cl. A61N 1/04

(52) U.S. Cl. 607/45

(57) ABSTRACT

An apparatus and method for electrostimulation treatment of neurological diseases is disclosed herein. The apparatus and method include an array (22) of sub-micron (and sub-cell size) FET electrodes (24) that are capacitively coupled to nervous system elements (both neurons (50) and axons (66)) as a replacement for traditional metal shanks in both single- and multi-electrode(s) electrostimulation implantable devices. By using such an approach, significant improvements in selectivity, power consumption and biocompatibility can be achieved, as well as relying on mainstream IC manufacture techniques for the manufacture thereof, making it cost-effective. The present disclosure can also be extended to any application where capacitive coupling to single or multiple cells can be used for sensing and/or stimulation thereof.

The present disclosure relates generally to an array of sub-micron (e.g., sub-cell size) elements for the capacitive stimulation and/or detection of biological tissue. In particular, the present disclosure relates to an array of FET electrodes to sense and trigger neuron action with respect to neurological disease and, more particularly, to intracranial stimulation for optimal control of movement disorders and other neurological disease.

There are a wide variety of treatment modalities for neurological disease including movement disorders such as Parkinson's Disease, Huntington's Disease, and Restless Leg Syndrome, as well as psychiatric disease including depression, bipolar disorder and borderline personality disorders. These treatment modalities are moderately efficacious; however, they suffer from severe drawbacks.

One common conventional technique for controlling neurological disease includes the continuous electrical stimulation of a predetermined neurological region. Chronic high frequency intracranial electrical stimulation is typically used to inhibit cellular activity in an attempt to functionally replicate the effect of tissue ablation, such as pallidotomy and thalamotomy. Acute electrical stimulation and electrical recording and impedance measuring of neural tissue have been used for several decades in the identification of brain structures for both research purposes as well as for target localization during neurosurgical operations for a variety of neurological diseases. During intraoperative electrical stimulation, reduction in tremor has been achieved using frequencies typically on the order of 75 to 330 Hz. Based on these findings, chronically implanted constant-amplitude electrical stimulators have been implanted in such sites as the thalamus, subthalamic nucleus and globus pallidus.

Typically, an electrode of an implantable medical device consists of a single or multiple pins with an electrically active tip, such as the “Utah electrode” or “Utah probe”, for example. The electrically active tips are used for either recording of neural activity or stimulating it, for example, to eliminate the symptoms of neural disease, such as Parkinson's disease. In the treatment of Parkinson's disease, the electrode is implanted surgically deep in the brain of the patient and then used to deliver electrical stimulation to targeted areas in the brain that control movement, blocking the abnormal nerve signals that cause tremor and Parkinson's disease symptoms.

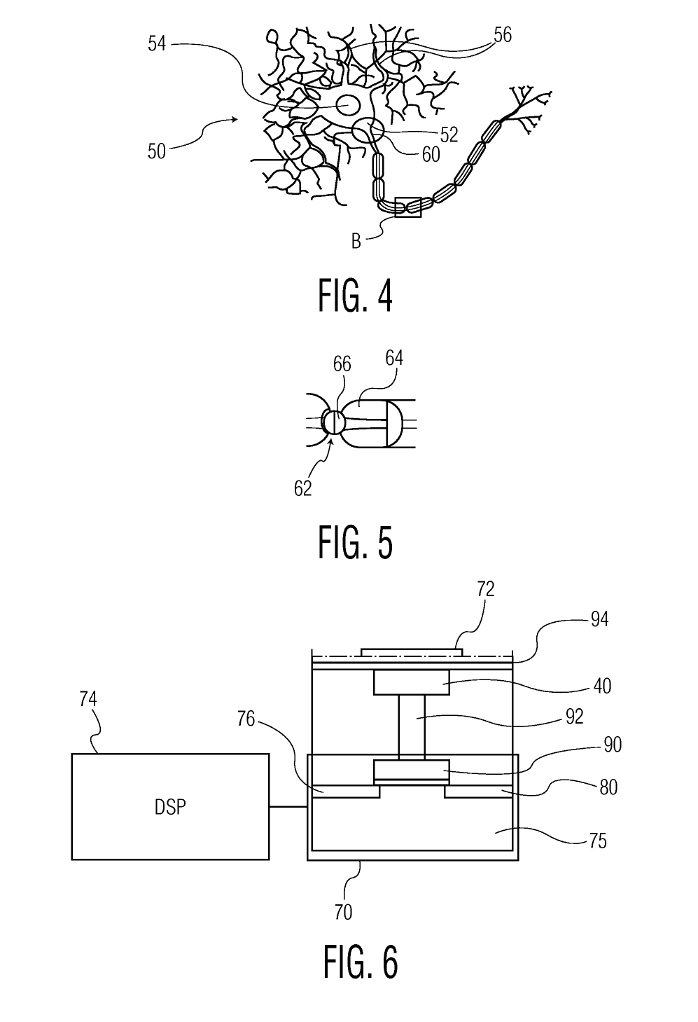

It is well accepted, that stimulation selectivity is of vital importance, both to improve the current applications (deep brain, but also other sorts of neurostimulation—peripheral nerves, vagus, sacral, cochlear, retina, etc.) as well as enabling future applications, not yet known. Ultimate selectivity will be achieved when it is possible to address a single neuron or neurite. Currently, employed electrodes cannot achieve this goal, either because of the size of the active element (typical mammal neuron dimensions are of the order of 10 μm as illustrated in FIG. 4) or the fact that only a limited number of electrodes can be inserted in the living tissue. Another issue with current approaches is that the stimulation usually involves dumping a large amount of electrical current into the tissue, which is effective in addressing large amounts of cells, but provides poor selectivity, and sometimes creates more damage to the cells than intended. Furthermore, this current approach consumes a significant amount of energy from the power source.

Thus, there is a need for an apparatus and method of electrostimulation/sensing in vivo that is effective in selectively addressing a large number of cells, without damaging the cells and limiting the power consumed to stimulate the tissue.

The present disclosure provides an apparatus for capacitive stimulation and/or detection of biological tissue for use in treating disease. In one embodiment, the apparatus includes a support structure; an array of at least one stimulation device and at least one sensing deice arranged in or on the support structure; and a dielectric layer having one layer surface and an opposite layer surface. The one layer surface is operably connected to the array and the opposite layer surface forms a stimulation and/or sensing surface for the capacitive stimulation and/or detection of biological tissue. Each stimulation device and sensing device is dimensioned as a sub-micron device in order to selectively address a single biological cell of the biological tissue.

The present disclosure also provides a method for capacitive stimulation and/or detection of biological tissue for use in treating disease. In one embodiment, the method includes: arranging an array of at least one stimulation device and at least one sensing deice on a Si substrate; and disposing a dielectric layer intermediate the array and biological tissue. The dielectric layer has one layer surface and an opposite layer surface. The one layer surface is operably connected to the array and the opposite layer surface forms a stimulation and/or sensing surface for the capacitive stimulation and/or detection of cells of the biological tissue. Each stimulation device and sensing device is dimensioned as a sub-micron device in order to selectively address a single biological cell of the biological tissue.

In exemplary embodiments, the support structure is a semiconductor structure, including for example a CMOS semiconductor structure having a field-effect transistor (FET) with a gate thereof used as the sensing and/or stimulation device.

In exemplary embodiments, the disease is a neurologic disease including one of Parkinson's disease, Huntington's disease, Parkinsonism, rigidity, hemiballism, choreoathetosis, dystonia, akinesia, bradykinesia, hyperkinesia, other movement disorder, epilepsy, or seizure disorder, for example.

Additional features, functions and advantages associated with the disclosed apparatus and method will be apparent from the detailed description which follows, particularly when reviewed in conjunction with the figures appended hereto.

To assist those of ordinary skill in the art in making and using the disclosed apparatus and method, reference is made to the appended figures, wherein:

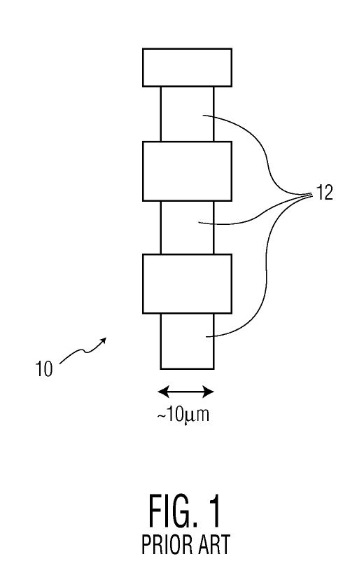

FIG. 1 is a top plan view of a shank having metal conductors that collect and inject currents in neural tissue to respectively sense and trigger neural activity as in the prior art;

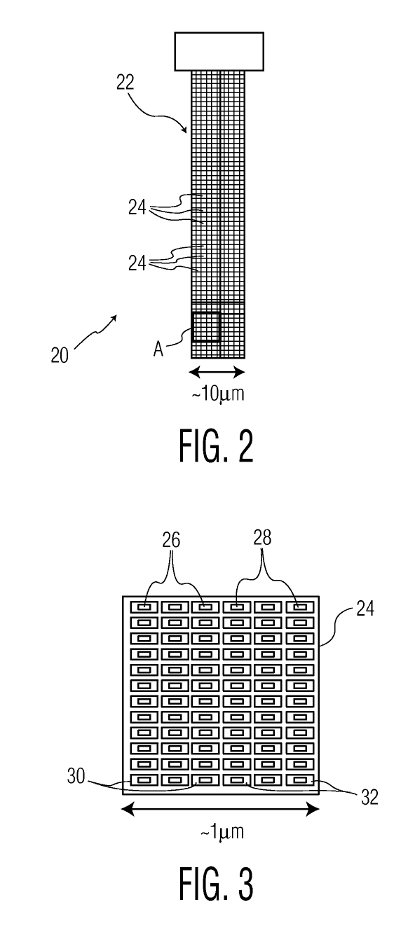

FIG. 2 is a top plan view of a shank having an array of FET electrodes that use capacitive coupling that sense and/or trigger and/or block propagation of neuron action or it's axon in accordance with an exemplary embodiment of the present disclosure;

FIG. 3 is an enlarged view of the inset of FIG. 2 illustrating sense and trigger FETs that are separately addressable in accordance with an exemplary embodiment of the present disclosure;

FIG. 4 illustrates a typical mammal neuron drawn to scale of the shank electrodes illustrated in FIG. 2;

FIG. 5 is an enlarged view of the inset of FIG. 4 illustrating nodes of Ranvier extending from the axon hillock of the neuron; and

FIG. 6 is an enlarged cross sectional view of a sensing/stimulation device operably connected to neural tissue and a digital signal processor (DSP) in accordance with an exemplary embodiment.

As set forth herein, the apparatus of the present disclosure advantageously permits and facilitates neural tissue interfacing, e.g., in implantable neurostimulation medical devices. The present disclosure can be extended to any application where capacitive coupling to single or multiple cells is desired for either sensing or stimulation thereof. More specifically, the present disclosure suggests using arrays of sub-micron (and sub-cell size) elements capacitively coupled to nervous system elements (e.g., both neurons and axons) that replace traditional use of metal shanks in both single- and multi-electrode(s) electrostimulation implantable devices. By using such an approach, significant improvements in selectivity, power consumption and biocompatibility can be achieved. Also, the apparatus of the present disclosure relies on mainstream IC manufacture techniques, making it cost-effective.

With reference to FIG. 1, a prior art shank electrode 10 is illustrated with each of the three metal conductors 12 having a dimension of about 10μ×10 μm. The metal conductors 12 are used to both sense and trigger neural activity collecting and injecting, respectively, electrical current in the neural tissue.

FIG. 2 illustrates an exemplary embodiment of a shank electrode 20 having an array 22 of field-effect transitior (FET) devices 24 (see FIG. 6) that replace the metal electrodes 12 of FIG. 1. It will be noted that the overall dimensions of the shank electrodes 10 and 20 are substantially the same. FIG. 3 illustrates an enlarged inset A of FIG. 2. FIG. 3 illustrates that the inset A of FIG. 2 is a square Si substrate 24 having a dimension of about 1 μm×1 μm. Substrate 24 includes a plurality of sensing devices 26 and a plurality of stimulation devices 28. More specifically, substrate 24 includes three columns 30 of sensing devices 26 and three columns 32 of stimulation devices 28. Columns 30 and 32 alternate with one another such that a sensing device 26 is proximate a stimulation device 28 in order to sense or trigger a current in an area defined by contiguous sensing and stimulation devices 26, 28. It will be recognized for example, but is not limited thereto, that substrate 24 of FIG. 3 illustrates thirty-six sensing and stimulation devices 26, 28 for a total of seventy-two devices in a 1 μm×1 μm area. Each device 26, 28 of FIG. 3 is a FET electrode illustrated as a metal contact 40 in FIG. 6.

Referring now to FIG. 4, a typical mammal neuron 50 is between about 1 μm to about 10 μm in diameter. FIG. 4 is drawn to the scale of FIG. 2 to illustrate the increased selectivity based on the number of devices 26, 28 of array 22 relative to neuron 50. FIG. 4 illustrates neuron 50 having a cell body 52, a cell nucleus 54 and dendrites 56 extending from the cell body 52. It is known that an optimum location to both sense and trigger action potential is the so-called “axon hillock” 60 with sub-micron dimensions. Inset B of FIG. 4 is enlarged in FIG. 5 illustrating “nodes of Ranvier” 62 where about 10 μm to about 20 μm parts of axon 66 are shown unprotected by myelin sheath 64.

As will be recognized, at least a pair of FET electrodes 26 and 28 is needed for sensing and stimulation, respectively. Better still, 4, 16 or more, as illustrated in FIGS. 2 and 3 will enable selection of the appropriate neuron 50 or population, even if neuron 50 moves via a digital signal processor (DSP), for example. Hence, deep sub-micron FET transistors are needed. The present disclosure suggests using standard Si technology, for example, 0.13 μm CMOS node technology as a possibility to have at least 16 devices 26, 28 per neuron 50.

Referring now to FIG. 6, a sensing device 26 is incorporated with a MOSFET 70, which is operably connected to neural tissue 72 and a signal processor 74, which can be implemented in a fast microprocessor, a DSP (digital signal processor) chip, or as analog circuitry, for example, is described in detail below. In an exemplary embodiment, signal processor 74 is a digital signal processor (DSP) 74. FIG. 6 illustrates a cross-section view of a sensing/stimulation device after processing. Although a sensing device is illustrated in FIG. 6, a stimulation device would have a gate electrode connected to a voltage terminal or be analogous to a floating gate electrode in nonvolatile memory (NVM) (e.g., carriers can be injected from the drain/source to create sufficient potential on the gate to trigger firing of neuron 50).

Sensing MOSFET 70 includes a substrate 75 having a source 76 and drain 80. In an exemplary embodiment, substrate 74 is a Si substrate used in standard Si technology.

FIG. 6 illustrates MOSFET 70 having a gate 90 of the sensing device used for sensing/stimulation. Gate 90 is connected by a low-loss metal wire 92 to a top surface dielectric layer 94 via metal contact 40. Dielectric layer 94 is used for actual coupling between electronics of MOSFET 70 and neural tissue 72.

The sensing/stimulation device of FIG. 6 is thus configured to sense/stimulate a cell body including similar considerations, including sensing/stimulation and even blocking (of the action potential propagations) action can be applied on “nodes of Ranvier” 62—e.g., 10-20 μm parts of axons 66 unprotected by myelin sheath 64.

As discussed above, the stimulation device preferably comprises a gate electrode 90, the electrical potential of which is externally controllable and the dielectric layer 94 is arranged on the metal contact 40, which is in contact with gate 90 via low-loss metal wire 92.

The support structure 75 preferably comprises a semiconductor structure. The semiconductor structure may be, in particular, a silicon CMOS structure.

The sensor device preferably comprises a FET with a source contact 76, a drain contact 80 and a gate contact 90. The FET may be, in particular, a p-transistor or a n-transistor, which is formed in the “front-end” of a CMOS process.

The dielectric layer 94 used according to the present disclosure is preferably arranged on the metal contact 40 of the sensor device which is connected in an electrically conductive manner to the gate contact of the field-effect transistor. The semiconductor structure is preferably a CMOS semiconductor structure. In particular, the metal electrode may be connected in an electrically conductive manner to the gate contact 90 via an arrangement of low-loss metal wires. The FET is also operably connected to the DSP 74 to selectively control/read each of the gate contacts 90 in array 22.

In one aspect of the invention, a neural modulation system for use in treating disease which provides stimulus intensity which may be varied is disclosed. The stimulation may be at least one of activating, inhibitory, and a combination of activating and inhibitory and the disease is at least one of neurologic and psychiatric. For example, the neurologic disease may include Parkinson's disease, Huntington's disease, Parkinsonism, rigidity, hemiballism, choreoathetosis, dystonia, akinesia, bradykinesia, hyperkinesia, other movement disorder, epilepsy, or the seizure disorder. The psychiatric disease may include, for example, depression, bipolar disorder, other affective disorder, anxiety, phobia, schizophrenia, multiple personality disorder. The psychiatric disorder may also include substance abuse, attention deficit hyperactivity disorder, impaired control of aggression, or impaired control of sexual behavior.

In another aspect of the invention, a neurological control system is disclosed. The neurological control system modulates the activity of at least one nervous system component, and includes at least one intracranial stimulating electrode, each constructed and arranged to deliver a neural modulation signal to at least one nervous system component; at least one sensor, each constructed and arranged to sense at least one parameter, including but not limited to physiologic values and neural signals, which is indicative of at least one of disease state, magnitude of symptoms, and response to therapy; and a stimulating and recording unit constructed and arranged to generate said neural modulation signal based upon a neural response sensed by said at least one sensor in response to a previously delivered neural modulation signal.

The disclosed apparatus optimizes the efficiency of energy used in the treatment given to the patient by minimizing to a satisfactory level the stimulation intensity to provide the level of treatment magnitude necessary to control disease symptoms without extending additional energy delivering unnecessary overtreatment and wasting energy. In present stimulation systems, a constant level of stimulation is delivered over a large area, resulting in either of two undesirable scenarios when disease state and symptoms fluctuate: (1) undertreatment, i.e. tremor amplitude exceeds desirable level or (2) overtreatment or excess stimulation, in which more electrical energy is delivered than is actually needed. In the overtreatment case, battery life is unnecessarily reduced. The energy delivered to the tissue in the form of a stimulation signal represents a substantial portion of the energy consumed by the implanted device; minimization of this energy substantially extends battery life, with a consequent extension of time in between reoperations to replace expended batteries. In addition, the apparatus of the present disclosure relies on mainstream IC manufacturing techniques providing a cost effective solution to the prior art.

Although the apparatus of the present disclosure has been described with reference to exemplary embodiments thereof, the present disclosure is not limited to such exemplary embodiments. Rather, the apparatus disclosed herein is susceptible to a variety of modifications, enhancements and/or variations, without departing from the spirit or scope hereof. Accordingly, the present disclosure embodies and encompasses such modifications, enhancements and/or variations within the scope of the claims appended hereto.

1. An apparatus for capacitive stimulation and/or detection of biological tissue for use in treating disease, the apparatus comprising:
a support structure (24);
an array (22) of at least one stimulation device (28) and at least one sensing deice (26) arranged in or on the support structure (24); and
a dielectric layer (94) having one layer surface and an opposite layer surface, the one layer surface is operably connected to the array (22) and the opposite layer surface forms a stimulation and/or sensing surface for the capacitive stimulation and/or detection of biological tissue (72);
wherein each stimulation device (28) and sensing device (26) is dimensioned as a sub-micron device in order to selectively address a single biological cell of the biological tissue (72).

2. The apparatus of claim 1, wherein the array (22) of at least one stimulation device (28) and at least one sensing deice (26) includes a plurality of each stimulation device (28) and sensing device (26) for the single biological cell.

3. The apparatus of claim 2, wherein the plurality of each stimulation device (28) and sensing device (26) includes between about 4 to about 16 devices per the single biological cell.

4. The apparatus of claim 1, wherein the stimulation device (28) includes a metal electrode (40), the electric potentional of which is externally controllable, and wherein the dielectric layer (94) is arranged on the metal electrode (40).

5. The apparatus of claim 1, wherein the support structure (24) includes a semiconductor structure.

6. The apparatus of claim 5, wherein the semiconductor structure is a CMOS semiconductor structure.

7. The apparatus of claim 5, wherein the CMOS semiconductor structure includes 0.13 μm CMOS node technology.

8. The apparatus of claim 5, wherein each stimulation device (28) and sensing device (26) each include a field-effect transistor (FET) with a source contact (76), a drain contact (80), and a gate contact (90).

9. The apparatus of claim 7, wherein the dielectric layer (94) is arranged on a metal electrode (40) of a MOSFET device (70) which is connected in an electrically conductive manner to the gate contact (90) of the field-effect transistor.

10. The apparatus of claim 9, wherein the metal electrode (40) is connected in an electrically conductive manner to the gate contact (90) via an arrangement of low-loss metal wires (92).

11. The apparatus of claim 10, wherein the dielectric layer (94) is connected to a neural cell (50).

12. The apparatus of claim 8, wherein the FET includes a floating-gate FET used for capacitive coupling to stimulate generation of an action potential or block a propagation of an action potential along an axon (66).

13. The apparatus of claim 1, wherein the stimulation device (28) provides stimulation to the biological tissue (72) for at least one of activating, inhibitory, and a combination of activating and inhibitory.

14. The apparatus of claim 1, wherein the disease is at least one of neurologic and psychiatric.

15. The apparatus of claim 1, wherein the neurologic disease includes at least one of Parkinson's disease, Huntington's disease, Parkinsonism, rigidity, hemiballism, choreoathetosis, dystonia, akinesia, bradykinesia, hyperkinesia, other movement disorder, epilepsy, or the seizure disorder.

16. The apparatus of claim 15, wherein said psychiatric disease includes at least one of depression, bipolar disorder, other affective disorder, anxiety, phobia, schizophrenia, multiple personality disorder.

17. The apparatus of claim 14, wherein the psychiatric disorder includes substance abuse, attention deficit hyperactivity disorder, impaired control of aggression, or impaired control of sexual behavior.

18. The apparatus of claim 1, further comprising:
a digital signal processor (DSP) (74) in operable communication with the array (22), the DSP (74) providing selective addressing of each stimulation device (28) and sensing device (26) of the array (22).

19. The apparatus of claim 1, wherein the array (22) is disposed on a shank, the shank being a three dimensional electrode shank having the array (22) disposed on at least two active surfaces of the shank to increase a number of selected biological cells.

20. A method for capacitive stimulation and/or detection of biological tissue for use in treating disease, the method comprising:
arranging an array (22) of at least one stimulation device (28) and at least one sensing device (26) on a Si substrate (24); and
disposing a dielectric layer (94) intermediate the array (22) and biological tissue (72), the dielectric layer (94) having one layer surface and an opposite layer surface, the one layer surface is operably connected to the array (22) and the opposite layer surface forms a stimulation and/or sensing surface for the capacitive stimulation and/or detection of cells of the biological tissue (72);
wherein each stimulation device (28) and sensing device (26) is dimensioned as a sub-micron device in order to selectively address a single biological cell of the biological tissue (72).
